(19) United States
(12) Patent Application Publication
(10) Pub. No.: US 2019/0000782 A1
(43) Pub. Date: Jan. 3, 2019

(54) COMBINATION MEDICATION FOR NEURO-DEGENERATIVE DISEASES AND SIDE-EFFECTS ASSOCIATED WITH COGNITION EFFECTING PHARMACEUTICALS

(71) Applicants: Cary Erwin Fechter, North Charleston, SC (US); Nancy Sharon Kedem, Melville, NY (US); Douglas F. Crane, Charleston, SC (US); Thomas James Thomas, Summerville, SC (US)

(72) Inventors: Cary Erwin Fechter, North Charleston, SC (US); Nancy Sharon Kedem, Melville, NY (US); Douglas F. Crane, Charleston, SC (US); Thomas James Thomas, Summerville, SC (US)

(21) Appl. No.: 16/125,124

(22) Filed: Sep. 7, 2018

Related U.S. Application Data
(63) Continuation-in-part of application No. 15/738,357, filed on Dec. 20, 2017.
Application No. 15/738,357 is a continuation of application No. 14/745,400, filed on Jun. 20, 2015, now abandoned.

(51) Int. Cl. A61K 31/145; A61K 31/343; A61K 45/06; A61K 31/135; A61K 31/137; A61K 31/445; A61K 31/165

(52) U.S. Cl. CPC A61K 31/145; A61K 2300/00; A61K 31/343; A61K 45/06; A61K 31/137; A61K 31/445; A61K 31/165; A61K 31/135

(57) ABSTRACT

The disclosure generally relates to pharmaceutical compositions including a benzhydrylsulfinylacetamide antidepressant and an amount of sodium chloride effective to increase a mean arterial blood pressure of a subject and methods for ameliorating symptoms of dementia including forms of dementia diagnosed as Alzheimer's disease and side-effects associated with the administration of cognition effecting pharmaceuticals.

### CROSS-REFERENCE TO RELATED APPLICATIONS

This application is a continuation-in-part of U.S. patent application Ser. No. 15/738,357 filed Dec. 20, 2017, which is a U.S. national phase of PCT Application No. PCT/US2016/038351 filed Jun. 20, 2016, which claims priority to U.S. patent application Ser. No. 14/745,400 filed Jun. 20, 2015, the disclosures of which are incorporated in their entirety by reference herein.

### BACKGROUND OF THE INVENTION

### Field of the Invention

The disclosure generally relates to pharmaceutical compositions including a benzhydrylsulfinylacetamide antidepressant and an amount of sodium chloride effective to increase a mean arterial blood pressure of a subject and methods for ameliorating symptoms of dementia including forms of dementia diagnosed as Alzheimer's disease and side-effects associated with the administration of cognition effecting pharmaceuticals.

### Description of the Related Art

Dementia is a common characteristic associated especially with increasing age and is sometimes referred to as “senility.” It is characterized by long term and generally gradual impairment of the ability to think and remember that is great enough to affect a person's daily functioning. Other common symptoms include emotional problems, decrease in language skills, paranoia, and decreased motivation. In 1906 Dr. Alois Alzheimer first recognized the disease now bearing his name. He described the disease as a dementia characterized by severe memory loss and confusion with pathological changes to neurons. Frequency of diagnosis increased with the implementation of a cognitive measurement scale in 1968 and currently Alzheimer's disease is the most widely diagnosed form of dementia. Understanding of the molecular basis of the disease improved with the discovery of the beta amyloid and tau proteins that form the toxic deposits causing neuron death. In spite of this, no curative agents have been developed although several acetylcholinesterase antagonists have been approved for treating memory deterioration. These drugs are currently being used with limited results. Common treatments for dementia include the use of antidepressants and anti-anxiety medications. However, these have proven to be only of marginal effectiveness as compared with placebos. Treatment for Alzheimer's disease is much less developed, as only somewhat recently were animal models proposed to test drug efficacy. A great deal of developmental work has centered on slowing the course of the disease. Little progress has been made in alleviating symptoms such as reduced cognitive behavior. It would be desirable to provide a pharmaceutical composition which is effective in improving the daily functioning of patients suffering from dementia in general, and also in patients diagnosed with Alzheimer's disease.

As related to cognition effecting pharmaceuticals, forms of poppyseed analgesics have been used for centuries but morphine itself was isolated in 1803. By 1850, it was being used medically since hypodermic needles allowed rapid delivery. Medicinally, morphine is commonly used for severe pain associated with traumatic injuries and cancer management. In cancer management there is a trade-off of thinking ability or cognition for pain relief which greatly reduces patients' ability to communicate with their family as cancer progresses. Thus, it would be desirable to provide a pharmaceutical composition improves patients' abilities to communicate.

### SUMMARY OF THE INVENTION

It has now been surprisingly and unexpectedly discovered that a therapy employing a combination of a benzhydrylsulfinylacetamide wakefulness promoting agent and either or both of a selective serotonin reuptake inhibitor (SSRI) and a stimulating antidepressant, and optionally a cholinesterase inhibitor, is effective in reducing symptoms associated with dementia. It has also been surprisingly and unexpectedly discovered that a therapy employing a combination of a benzhydrylsulfinylacetamide wakefulness promoting agent and an amount of sodium chloride effective to increase a mean arterial blood pressure of a subject is effective in reducing symptoms associated with dementia and side-effects such as sleepiness, lethargy, impaired cognition, or abnormal circadian rhythms associated with the administration of cognition effecting pharmaceuticals.

In various embodiments are disclosed compositions including a benzhydrylsulfinylacetamide wakefulness promoting agent and an amount of sodium chloride effective to increase a mean arterial blood pressure of a subject.

In various embodiments are disclosed methods for alleviating symptoms associated with dementia including administering a benzhydrylsulfinylacetamide wakefulness promoting agent and an amount of sodium chloride to a subject having dementia, wherein the amount of sodium chloride is effective to increase mean arterial blood pressure of the subject.

In various embodiments are disclosed methods for alleviating side-effects associated with the administration of a cognition effecting pharmaceutical including administering a benzhydrylsulfinylacetamide wakefulness promoting agent and an amount of sodium chloride to a subject having a side-effect from a cognition effecting pharmaceutical, wherein the amount of sodium chloride is effective to increase mean arterial blood pressure of the subject.

### DETAILED DESCRIPTION OF THE PREFERRED EMBODIMENTS

As required, detailed embodiments of the present disclosure are disclosed herein; however, it is to be understood that the disclosed embodiments are merely exemplary and may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art.

Except in the examples, or where otherwise expressly indicated, all numerical quantities in this description indicating amounts of material or conditions of reaction and/or use are to be understood as modified by the word “about”. The first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

Unless indicated otherwise, all technical and scientific terms used herein have the same meaning as commonly understood by one of ordinary skill in the art to which the present disclosure belongs.

It is also to be understood that this disclosure is not limited to the specific embodiments and methods described below, as specific components and/or conditions may, of course, vary. Furthermore, the terminology used herein is used only for describing particular embodiments and is not intended to be limiting in any way.

It is also noted that, as used in the specification and the appended claims, the singular form “a,” “an,” and “the” comprise plural referents unless the context clearly indicates otherwise. For example, reference to a component in the singular is intended to comprise a plurality of components.

The term “or” can be understood to mean “at least one of”. The term “and” can also be understood to mean “at least one of” or “all”.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

The terms “comprising”, “consisting of”, and “consisting essentially of” can be alternatively used. When one of these three terms is used, the presently disclosed and claimed subject matter can include the use of either of the other two terms.

The term “side-effect(s)” refers to any abnormality, adverse effect, unwanted effect, or secondary effect that may be caused by taking a pharmaceutical or drug such as a cognition effecting pharmaceutical. Examples of a side-effect can include sleepiness, lethargy, impaired cognition, or abnormal circadian rhythms.

The term “effective amount” of a drug, compound, pharmaceutical, pharmaceutical composition, or composition is an amount sufficient to effect beneficial or desired results. For example, an effective amount can include amounts used for reducing symptoms associated with dementia and side-effects such as sleepiness, lethargy, impaired cognition, or abnormal circadian rhythms associated with the administration of cognition effecting pharmaceuticals. The term “compound(s)” and “pharmaceutical(s)” can be used interchangeably to refer to a compound or pharmaceutical composition. The terms “drug(s)”, “compound(s)”, “pharmaceutical(s)”, “pharmaceutical composition”, or “composition(s)” are be used interchangeably.

The term “subject(s)” or “patient(s)” are used interchangeably to refer to a subject of any mammalian species such as, but not limited to, humans, dogs, cats, horses, rodents, any domesticated animal, or any wild animal.

The term “mean arterial pressure” or “MAP” refers to the average arterial pressure during a single cardiac cycle.

Our research has revealed that two symptoms associated with Alzheimer's disease as well as other neurodegenerative diseases but not addressed by current therapeutic regimens, specifically depression and overall abnormal sleep patterns, can have a significant negative effect on the quality of life of patients. The abnormal sleep patterns in particular, by disrupting normal circadian rhythm, exacerbate the confusion, loss of mental focus, and feelings of disorientation characteristic of the disease. Furthermore, this abnormal circadian pattern of daytime sleepiness combined with restlessness and inability to sleep properly at night (sometimes referred to as “sundowning”) greatly complicates the efforts of caregivers.

Including treatment for these two symptoms in the overall treatment protocol can improve memory and patient quality of life as much as the drugs currently approved for memory deterioration.

Since traditional sleep regulating drugs such as tricyclics and benzodiazepines can increase the lethargy, confusion, and forgetfulness, already present in Alzheimer's patients, a different therapeutic approach is needed. Specifically, drugs of the benzhydrylsulfonylamide type can reduce excessive daytime sleepiness and promote normal circadian rhythm while avoiding the side effects of traditional sleep regulating drugs.

Combining this therapy with administration of either or both of a Selective Serotonin Reuptake Inhibitor (SSRI) antidepressant and a stimulating antidepressant such as bupropion reduces the severity of associated clinical depression symptoms. SSRI's also increase alpha secretase activity, which has been shown to aid in clearing amyloid beta, one of the peptides associated with formation of damaging plaques in the brain.

Combining the drugs of the benzhydrylsulfonylamide type therapy with administration of an amount of sodium chloride effective to increase a mean arterial blood pressure of a subject also reduces the severity of associated clinical depression symptoms and sleepiness or lethargy associated with the administration of cognition effecting pharmaceuticals.

These combination therapies are also able to alleviate side-effects associated with the administration of cognition effecting pharmaceutical.

The overall results of this combination approach in conjunction with traditional acetylcholinesterase antagonist therapy are improvements in patient cognition including responsiveness and attitude toward their environment.

Additionally, combining all of the active pharmaceutical agents in a single formulation ensures that the timing of administration of the drugs is optimal. Since Alzheimer's caregivers typically have multiple duties to their patients, the single dosage format simplifies caregiver routine and improve compliance with the treatment regimen.

In various embodiments are disclosed compositions including a benzhydrylsulfinylacetamide wakefulness promoting agent and an amount of sodium chloride effective to increase a mean arterial blood pressure of a subject. The compositions of various embodiment can further include one of a cognition effecting pharmaceutical, selective serotonin reuptake inhibitor, stimulating antidepressant, cholinesterase inhibitor, cognition effecting compound, or combinations thereof. In other embodiments, the compositions can further include a concentration of a compound effective for increasing a subject's MAP by 10 millimeter of mercury (mm Hg) or less.

In various embodiments are disclosed methods for alleviating symptoms associated with dementia including administering a benzhydrylsulfinylacetamide wakefulness promoting agent and an amount of sodium chloride to a subject having dementia, wherein the amount of sodium chloride is effective to increase mean arterial blood pressure of the subject.

In various embodiments are disclosed methods for alleviating side-effects associated with the administration of a cognition effecting pharmaceutical including administering a benzhydrylsulfinylacetamide wakefulness promoting agent and an amount of sodium chloride to a subject having a side-effect from a cognition effecting pharmaceutical, wherein the amount of sodium chloride is effective to increase mean arterial blood pressure of the subject.

Benzhydrylsulfinylacetamides of various embodiments are known, as described in Lafon EP 0097071, and correspond to the general formula

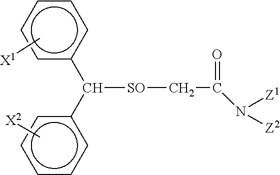

wherein

X¹ and X² may be the same or different, and are H, Cl, or F;

Z¹ and Z² may be the same or different and are H, —CH₃, —CH(CH₃)₂, or —C(CH₃)₃, with the proviso that at least one of Z¹ and Z² is H.

Preferably, both Z¹ and Z² are H, and also preferably one of the following holds: X¹ and X² are both H, one of X¹ and X² is H and the other of X¹ and X² is 4-Cl or 4-F, or both X¹ and X² are 4-F. Most preferably, both Z¹ and Z² are H and both X¹ and X² are H, in other words, benzhydrylsulfinylacetamide itself (CAS 68693-11-8). The benzhydrylsulfinylacetamides may be used in racemic form, or in the form of their fully or partially resolved optical isomers wherein one isomer, preferably the levorotatory form, is present in enantiomeric excess. Racemic benzhydrylsulfinylacetamide is available commercially as PROVIGIL® from Cephalon, Inc., and is known pharmaceutically as modafinil.

The sodium chloride of various embodiments includes pharmaceutically-acceptable salts useful in the regulation of the cardiovascular system. In various embodiments, the amount of sodium chloride effective to increase a mean arterial blood pressure of a subject is or is at least 0.25 grams (g), 0.3 g, 0.35 g, 0.4 g, 0.45 g, 0.5 g, 0.55 g, 0.6 g. 0.65 g. 0.7 g, 0.75 g, 0.8 g, 0.85 g, 0.9 g, 0.95 g, 1 g, 1.05 g, 1.1 g, 1.15 g, 1.2 g, 1.25 g, 1.3 g, 1.35 g, 1.4 g, 1.45 g, 1.5 g, 1.55 g, 1.6 g, 1.65 g, 1.7 g, 1.75 g, 1.8 g, 1.85 g, 1.9 g, 1.95 g, 2 g, 2.05 g, 2.1 g, 2.15 g, 2.2 g, 2.25 g, 2.3 g, 2.35 g, 2.4 g, 2.45 g, 2.5 g, 2.55 g, 2.6 g, 2.65 g, 2.7 g, 2.75 g, 2.8 g, 2.85 g, 2.9 g, 2.95 g, 3 g, 3.05 g, 3.1 g, 3.15 g, 3.2 g, 3.25 g, 3.3 g, 3.35 g, 3.4 g, 3.45 g, 3.5 g, 3.55 g, 3.6 g, 3.65 g, 3.7 g, 3.75 g, 3.8 g, 3.85 g. 3.9 g, 3.95 g, 4 g, 4.05 g, 4.1 g, 4.15 g, 4.2 g, 4.25 g, 4.3 g, 4.35 g, 4.4 g, 4.45 g, 4.5 g, 4.55 g, 4.6 g, 4.65 g, 4.7 g, 4.75 g, 4.8 g, 4.85 g, 4.9 g, 4.95 g, or 5 g. In various embodiments, the amount of sodium chloride effective to increase a mean arterial blood pressure of a subject is a range between any two amounts listed above.

In various embodiments, the amount of sodium chloride when administered to the subject increases the subject's MAP by 10 mm Hg or less. In other embodiments, the amount of sodium chloride when administered to the subject increases the subject's MAP by 0.5 mm Hg, 1 mm Hg, 1.5 mm Hg, 2 mm Hg, 2.5 mm Hg, 3 mm Hg, 3.5 mm Hg, 4 mm Hg, 4.5 mm Hg, 5 mm Hg, 5.5 mm Hg, 6 mm Hg, 6.5 mm Hg, 7 mm Hg, 7.5 mm Hg, 8 mm Hg, 8.5 mm Hg, 9 mm Hg, 9.5 mm Hg, or 10 mm Hg. In various embodiments, increase in the subject's MAP is a range between any two pressures listed above.

The compositions of various embodiments can further include an amount of a compound effective for increasing a subject's MAP by 10 mm Hg or less. In other embodiments, the compositions can further include an amount of a compound effective for increasing a subject's MAP by 0.5 mm Hg, 1 mm Hg, 1.5 mm Hg, 2 mm Hg, 2.5 mm Hg, 3 mm Hg, 3.5 mm Hg, 4 mm Hg, 4.5 mm Hg, 5 mm Hg, 5.5 mm Hg, 6 mm Hg, 6.5 mm Hg, 7 mm Hg, 7.5 mm Hg, 8 mm Hg, 8.5 mm Hg, 9 mm Hg, 9.5 mm Hg, or 10 mm Hg. In various embodiments, increases in the subject's MAP is a range between any two pressures listed above.

The compositions of various embodiments can include amounts of sodium chloride and a MAP increasing compound that together are effective for increasing a subject's MAP by 10 mm Hg or less. In other embodiments, the combination of the amounts of the sodium chloride and the compound are effective for increasing a subject's MAP by 0.5 mm Hg, 1 mm Hg, 1.5 mm Hg, 2 mm Hg, 2.5 mm Hg, 3 mm Hg, 3.5 mm Hg, 4 mm Hg, 4.5 mm Hg, 5 mm Hg, 5.5 mm Hg, 6 mm Hg, 6.5 mm Hg, 7 mm Hg, 7.5 mm Hg, 8 mm Hg, 8.5 mm Hg, 9 mm Hg, 9.5 mm Hg, or 10 mm Hg, In various embodiments, increases in the subject's MAP is a range between any two pressures listed above.

Examples of compounds that can increase a subject's MAP includes anabolic steroids such as norandrolone, oxymetholone, oxandrolone; antidepressants such as venlafaxine, tricyclics, and monoamine oxidase inhibitors; antiobesity drugs such as Sibutramine and phentermine; cholesteryl ester transfer protein inhibitors such as torcetrapib; erythropoietin such as rHuEPO and darbepoetin; herbal preparations such as ephedra; immunosuppressants such as cyclosporin and tacrolimus; mineralocorticoids such as licorice, carbenoxolone, fludrocortisone. 9α-fluoroprednisolone; Nonsteroidal anti-inflammatories or coxibs such as all inhibitors with COX-2 increasing greater than COX-1 and indomethacin; oral contraceptives such as estrogen containing serotonergics (antimigraine), dihydroergotamine, and sumatriptan; sulfonylureas such as glibenclamide; or sympathomimetic amines such as catecholamines and analogs and phenylpropanolamine.

In alternative embodiments, compound effective for increasing a subject's MAP by 10 mm Hg or less can be substituted for the sodium chloride.

Selective serotonin reuptake inhibitors (SSRIs) of various embodiments, also known as “serotonin-specific reuptake inhibitors” increase the extracellular concentration of the neurotransmitter serotonin. The action of SSRIs may be contrasted with serotonin reuptake inhibitors (SRIs), as the latter also greatly affect reuptake of dopamine and norepinephrine. SSRIs primarily affect serotonin reuptake, with lesser effect on the two other neurotransmitters, e.g. are “selective” or “specific” relative to reuptake of serotonin. By preventing serotonin reuptake, believed to be caused by minimizing reabsorbtion of serotonin into presynaptic cells, extracellular serotonin levels are increased.

Those skilled in the pharmaceutical arts are aware of numerous SSRIS which can be used. Unlike the benzhydrylsulfinylacetamides, the SSRIs span a wide range of chemical structures which share little in common except their serotonin selectivity. Useful SSRIs and/or their salts include citalopram (CAS 59729-33-8), escitalopram (CAS 128196-01-1), fluoxetine (CAS 54910-89-3), fluvoxamine (CAS 56296-78-7), paroxetine (CAS 78246-49-8), and sertraline (CAS 79617-96-2). Preferred are citalopram, escitalopram, fluoxetine, paroxetine, sertraline. More preferred are citalopram and sertraline. Sertraline is most preferred.

Stimulating antidepressants of various embodiments as defined therein are dopamine and norepinephrine reuptake inhibitors, and are associated with a decrease in depression symptoms along with an increased energy level. Bupropion is a stimulating antidepressant, and is preferred. Stimulating depressants as used herein do not include SSRIs, which have little effect on dopamine or norepinephrine reuptake.

A cholinesterase inhibitor of various embodiments is an optional ingredient. Cholinesterase inhibitors are known, examples of which include donepezil (CAS 120014-06-4), galantamine, and rivastigmine. Donepezil is preferred. The cholinesterase inhibitor may include more than one such inhibitor. Examples of combinations include donepezil and galantamine, donepezil and rivastigmine, donepezil, galantamine, and rivastigmine, and galantamine and rivastigmine.

A cognition effecting pharmaceutical or cognition effecting compound is a compound that can affect sleep, mood, cognition, or a circadian rhythm of a patient or subject after administration. Examples of cognition affecting pharmaceuticals can include opioids, depressants, sedatives, hallucinogens, anxiolytics, or anesthetics. Opiates, are a class of centrally acting compounds and are frequently used agents for pain control. Opiates are narcotic agonistic analgesics and are drugs derived from opium, such as morphine, codeine, and many synthetic congeners of morphine, with morphine being the most widely used derivative. Opioids are natural and synthetic drugs with morphine-like actions and include the opiates. Opioids are narcotic agonistic analgesics which produce drug dependence of the morphine type and are subject to control under federal narcotics law because of their addicting properties. The chemical classes of opioids with morphine-like activity are the purified alkaloids of opium consisting of phenanthrenes and benzylisoquinolines, semi-synthetic derivatives of morphine, phenylpiperidine derivatives, morphinan derivatives, benzomorphan derivatives, diphenyl-heptane derivatives, and propionanilide derivatives. The principal phenanthrenes are morphine, codeine, and thebaine. The principal benzoisoquinolines are papaverine, a smooth muscle relaxant, and noscapine. Semi-synthetic derivatives of morphine include diacetylmorphine (heroin), hydromorphone, oxymorphone, hydrocodone, apomorphine, etorpine, and oxycodone. Phenylpiperidine derivatives include meperidine and its congeners diphenoxylate and loperamide, alphaprodine, anileridine hydrochloride or phosphate, and piminodine esylate. Morphinan derivatives include levorphanol. The diphenyl-heptane derivatives include methadone and its congeners, and propoxyphene. Propionanilide derivatives include fentanyl citrate and its congeners sufenil citrate and alfenatil hydrochloride. Depressants include, but are not limited to, nonbarbiturates, methaqualone, barbiturates, diazepam, flurazepam, phencyclidine and fluoxetine. Examples of anxiolytics include alprazolam, bromazepam, chlordiazepoxide, clonazepam, clorazepate, diazepam, flurazepam, lorazepam, oxazepam, temazepam, or triazolam. Examples of anesthetics include ketamine, benzodiazepines, barbiturates, propofol, or etomidate.

The CAS numbers previously given may be those of the active ingredient base compound itself, or a pharmaceutically acceptable salt thereof. Those skilled in the art are cognizant of numerous counterions which can be used in such pharmaceutically acceptable salts. These salts are used in particular to alter the solubility or solubility profile of the particular compound. Suitable salts, e.g., are the hydrohalide salts such as hydrochlorides and hydrobromides, acetates, propionates, maleates, oxalates, bezylates, nitrates, sulfates, phosphates, and tartrates. This list is exemplary and not limiting. Further examples of pharmaceutically acceptable salts may be found in P. H. Stahl and C. G. Wermuth, Handbook of Pharmaceutical Salts: Properties, Wiley-VCH, Weinheim, 2002.

The amounts of each individual ingredient are therapeutically effective amounts, which can be varied depending on the individual patient's body chemistry. The ingredients are preferably contained in a single dosage, for example a pill, capsule, lozenge, gel cap, etc. The therapeutically effective amounts can be determined in a conventional manner, for example using double blind testing. In no case should any ingredient be present in an amount generally considered toxic.

As guidance to one skilled in the arts of pharmaceutical compounding and clinical trials, the benzhydrylsulfinylacetamide dosage is preferably in the range of 50-500 mg, more preferably 100-300 mg. A dosage of 200 mg of modafinil has been found to be particularly effective. Other benzhydrylsulfinylacetamides can be used in similarly effective amounts, meaning that compounds from this class which are more active may be used in correspondingly lesser amounts, and vice versa.

The SSRI antidepressant is preferably used in amounts of from 5 to 250 mg, more preferably 50-200 mg, and most preferably, 75-150 mg. A dosage of 100 mg of sertraline has been found to be particularly effective. SSRIs which are more active than sertraline can be used in lesser quantities, and vice versa. Citalopram is preferably used in amounts of from 5-50 mg.

The cholinesterase inhibitor, when included, is preferably used in amounts of from 1.5 to 20 mg, more preferably 3-15 mg, yet more preferably 3-10 mg, and most preferably from 3-8 mg. A dosage of 5 mg of donepezil has been shown to be particularly effective. For cholinesterase inhibitors with greater activity, correspondingly lesser dosages can be used, and vice versa.

The stimulating antidepressant is preferably bupropion, and when included, is preferably used in amounts of 20 mg to 400 mg, more preferably 50-250 mg, and most preferably 75-200 mg. A dosage of 150 mg has been proven to be particularly effective. For stimulating antidepressants of higher activity, correspondingly lesser amounts may be used, and vice versa.

Each of the ranges given for each of the flour classes of ingredients, benzhydrylsulfinylacetamides, SSRI inhibitors, cholinesterase inhibitors, and stimulating depressants, may be used in conjunction with any of the ranges of the other classes of ingredients. This allows the formulation to be altered for specific patients or for specific classes of patients with similar body chemistry. Moreover, each start point and end point of each range for any one class of ingredients may be combined with an end point or start point of another disclosed range for the same ingredient or ingredient class to define another range.

The quantities given above are intended for simultaneous administration once per day. If administration is to be performed more than once per day, it is preferred that the dosages be lowered. It is preferable that administration be once per day in the morning. A second dose at midday or in the early late afternoon may also be used. It is not preferable that administration take place in late afternoon or evening, as the ability to achieve a restful night's sleep may be impaired.

The compositions of the subject invention may be formulated in liquid, gel, or solid form, using conventional pharmaceutical excipients. Solid dosage forms are preferred. Non-limiting examples of suitable excipients include fillers, examples of which are dextrose, sucrose, starches, and calcium carbonate; tableting aids such as metal stearates; coatings such as polyvinylpyrrolidone, polyvinylalcohol, and crosslinked and uncrosslinked gelatin; thickeners such as modified celluloses, for example carboxymethyl cellulose; natural oils and modified natural oils such as fatty acid esters, olive oil, fish oil, liquid and solid triglycerides, and the like. Suitable pharmaceutical excipients may be found in numerous treatises, for example, A. H. Kibbe, Handbook of Pharmaceutical Excipients, APhA publications, ©2000.

The symptom relieving effects of the claimed compositions was evaluated by clinical observation of patients exhibiting symptoms of dementia, including patients diagnosed with Alzheimer's disease.

### EXAMPLES

### Example 1

A 50 year old female with forgetfulness, previously diagnosed with mild Alzheimer's disease by a neurologist was evaluated by questionnaire using the Epworth Sleepiness Scale (ESS) and found to be exhibiting excessive daytime sleepiness. Patient responses were consistent with clinical depression using the Beck Depression Scale (BDS) as characterized in the Diagnostic and Statistical Manual, fifth edition (DSM5).

A treatment protocol consisting of simultaneously administering 200 mg. Modafinil, 100 mg. Sertraline, and 5 mg. Donepezil once per day in the morning was initiated.

After six weeks the patient was re-evaluated by questionnaire using the ESS and found to be exhibiting a more normal sleep pattern. Patient responses demonstrated lessened depression criteria using BDS/DSM5.

Patient described overall attitude as more positive.

### Example 2

A 65 year old female with forgetfulness, previously diagnosed with moderate-severe Alzheimer's disease by a neurologist was evaluated through caregiver responses using the ESS and found to be exhibiting excessive daytime sleepiness.

Caregiver responses to standard questionnaire provided positive indication of clinical depression using BDS/DSM5.

A treatment protocol consisting of simultaneously administering 200 mg. Modafinil, 100 mg. Sertraline, and 5 mg. Donepezil once per day in the morning was initiated.

After six weeks the patient was re-evaluated by questionnaire through caregiver's responses using the ESS and found to be exhibiting a more normal sleep pattern. Caregiver responses demonstrated lessened patient depression criteria using BDS/DSM5.

Caregiver described patient's overall attitude as more positive.

### Example 3

A 55 year old male with forgetfulness, previously diagnosed with mild Alzheimer's disease by a neurologist was evaluated by questionnaire using the Epworth Sleepiness Scale (ESS) and found to be exhibiting excessive daytime sleepiness. Patient responses were consistent with clinical depression using the Beck Depression Scale (BDS) as characterized in the Diagnostic and Statistical Manual, fifth edition (DSM5).

A treatment protocol consisting of simultaneously administering 200 mg. Modafinil, 100 mg. Sertraline, and 5 mg. Donepezil once per day in the morning was initiated.

After six weeks the patient was re-evaluated by questionnaire using the ESS and found to be exhibiting a more normal sleep pattern. Patient responses demonstrated lessened depression criteria using BDS/DSM5

Patient described overall attitude as more positive.

### Example 4

A 71 year old male with forgetfulness, previously diagnosed with moderate-severe Alzheimer's disease by a neurologist was evaluated through caregiver responses using the ESS and found to be exhibiting excessive daytime sleepiness.

Caregiver responses to standard questionnaire provided positive indication of clinical depression using, BDS/DSM5.

A treatment protocol consisting of simultaneously administering 200 mg. Modafinil, 100 mg. Sertraline, and 5 mg. Donepezil once per day in the morning was initiated.

After six weeks the patient was re-evaluated by questionnaire through caregiver's responses using the ESS and found to be exhibiting a more normal sleep pattern. Caregiver responses demonstrated lessened patient depression criteria using BDS/DSM5.

Caregiver described patient's overall attitude as more positive.

### Example 5

A 53 year old female with forgetfulness, previously diagnosed with mild Alzheimer's disease by a neurologist was evaluated by questionnaire using the Epworth Sleepiness Scale (ESS) and found to be exhibiting excessive daytime sleepiness. Patient responses were consistent with clinical depression using the Beck Depression Scale (BDS) as characterized in the Diagnostic and Statistical Manual, fifth edition (DSM5).

A treatment protocol consisting of simultaneously administering 200 mg. Modafinil, 100 mg. Sertraline, 5 mg. Donepezil, and 150 mg. Bupropion once per day in the morning was initiated.

After six weeks the patient was re-evaluated by questionnaire using the ESS and found to be exhibiting a more normal sleep pattern. Patient responses demonstrated lessened depression criteria using BDS/DSM5.

Patient described overall attitude as more positive.

### Example 6

A 65 year old female with forgetfulness, previously diagnosed with moderate-severe Alzheimer's disease by a neurologist was evaluated through caregiver responses using the ESS and found to be exhibiting excessive daytime sleepiness.

Caregiver responses to standard questionnaire provided positive indication of clinical depression using BDS/DSM5.

A treatment protocol consisting of simultaneously administering 200 mg. Modafinil 100 mg. Sertraline, 5 mg. Donepezil, and 150 mg. Bupropion once per day in the morning was initiated.

After six weeks the patient was re-evaluated by questionnaire through caregiver's responses using the ESS and found to be exhibiting a more normal sleep pattern. Caregiver responses demonstrated lessened patient depression criteria using BDS/DSM5.

Caregiver described patient's overall attitude as more positive.

### Example 7

A 50 year old male with forgetfulness, previously diagnosed with mild Alzheimer's disease by a neurologist was evaluated by questionnaire using the Epworth Sleepiness Scale (ESS) and found to be exhibiting excessive daytime sleepiness. Patient responses were consistent with clinical depression using the Beck Depression Scale SDS) as characterized in the Diagnostic and Statistical Manual, fifth edition (DSM5).

A treatment protocol consisting of simultaneously administering 200 mg. Modafinil, 100 mg. Sertraline. 5 mg. Donepezil, and 150 mg. Bupropion once per day in the morning was initiated.

After six weeks the patient was re-evaluated by questionnaire using the ESS and. found to be exhibiting a more normal sleep pattern. Patient responses demonstrated lessened depression criteria using BDS/DSM5.

Patient described overall attitude as more positive.

### Example 8

A 72 year old male with forgetfulness, previously diagnosed with moderate-severe Alzheimer's disease by a neurologist was evaluated through caregiver responses using the ESS and found to be exhibiting excessive daytime sleepiness.

Caregiver responses to standard questionnaire provided positive indication of clinical depression using BDS/DSM5.

A treatment protocol consisting of simultaneously administering 200 mg. Modafinil, 100 mg. Sertraline, 5 mg. Donepezil, and 150 mg. Bupropion once per day in the morning was initiated.

After six weeks the patient was re-evaluated by questionnaire through caregiver's responses using the ESS and found to be exhibiting a more normal sleep pattern. Caregiver responses demonstrated lessened patient depression criteria using BDS/DSM5.

Caregiver described patient's overall attitude as more positive.

### Example 9

REVHESIC-S, where the “S” refers to the sodium chloride concentration that supports blood pressure maintenance.

In different examples, the composition can include Modafinil and 1, 2, or 2.5 g of sodium chloride. Alternatively, the concentration of sodium chloride can from 0.5 to 3 grams of sodium chloride. The composition of various embodiments can be used to treat side effects of cognition effecting pharmaceuticals. For example, anxiolytics reduce anxiety and are the most common class of the Diazepams. The Modafinil in composition can reverse the sleepiness and reduced cognition associated with the use of analgesics and anxiolytics. In another example, the composition including modafinil and sodium chloride can be used to improve the cognition of patients who are using Fentanyl patches, any other narcotic in oral IV, or transdermal deliveries of the cognition affecting pharmaceuticals. It has been shown in limited clinical trials that Modafinil reverses the sleepiness and even confusion associated with morphine delivery. Furthermore, the three most common side effects of morphine lowered blood pressure, lowered respiratory efforts called respiratory suppression, and constipation are issues that can be addressed in combinations with modafinil. By far the easiest combined combination remedy would be for support of blood pressure. In the ICU setting, normal saline is frequently given. However in the home setting where transdermal Fentanyl patches are used, blood pressure support could be in the form of sodium chloride salt combined with the modafinil. Patients may also have comorbidities which will have various effects on blood pressure. Hence a range of salt support is listed. The most common comorbidities would be infection which would require a greater dose of sodium chloride. Also, commonly greater pain is associated with brain metastases or bone metastases. The greater the analgesic use the more likely blood pressure will be reduced. They are for these examples will require higher doses of fentanyl delivery and will require larger doses of salt support. Thus as proposed in different examples, the combination of modafinil and normal saline in preset doses can reduce the morbidity and extend life in patients requiring analgesics or sedatives.

As required, detailed embodiments of the present invention are disclosed herein; however, it is to be understood that the disclosed embodiments are merely exemplary of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A pharmaceutical composition, comprising:
a) a benzhydrylsulfinylacetamide wakefulness promoting agent; and
b) an amount of sodium chloride effective to increase a mean arterial blood pressure of a subject.

2. The pharmaceutical composition of claim 1 further comprising at least one of a selective serotonin reuptake inhibitor, stimulating antidepressant, cholinesterase inhibitor, or a cognition effecting compound.

3. The pharmaceutical composition of claim 1, wherein the amount of sodium chloride is 0.25 grams or more.

4. The pharmaceutical composition of claim 1, wherein the amount of sodium chloride is effective to raise the mean arterial blood pressure of the subject by 10 mm Hg or less.

5. The pharmaceutical composition of claim 1, wherein the benzhydrylsulfinylacetamide wakefulness promoting agent has the formula 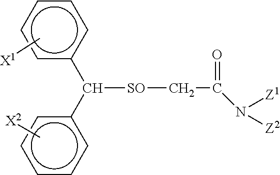
wherein
X¹ and X² may be the same or different, and are H, Cl, or F;
Z¹ and Z² are the same or different and are H, —CH₃, —CH(CH₃)₂, or —C(CH₃)₃, with the proviso that at least one Z¹ and Z² is H, or a pharmaceutically acceptable salt thereof.

6. The pharmaceutical composition of claim 1, wherein the benzhydrylsulfinylacetamide wakefulness promoting agent is benzhydrylsulfinylacetamide, or a pharmaceutically acceptable salt thereof.

7. The pharmaceutical composition of claim 1, wherein the benzhydrylsulfinylacetamide wakefulness promoting agent is present in an amount of 50-500 mg.

8. A method for alleviating symptoms associated with dementia comprising:
administering a benzhydrylsulfinylacetamide wakefulness promoting agent and an amount of sodium chloride to a subject having dementia;
wherein the amount of sodium chloride is effective to increase mean arterial blood pressure of the subject.

9. The method of claim 8, wherein the dementia is diagnosed as Alzheimer's disease.

10. The method of claim 8, wherein the amount of sodium chloride is 0.25 grams or more.

11. The method of claim 8, wherein the amount of sodium chloride is effective to raise the mean arterial blood pressure of the subject by 10 mm Hg or less.

12. The method of claim 8, wherein the benzhydrylsulfinylacetamide wakefulness promoting agent has the formula 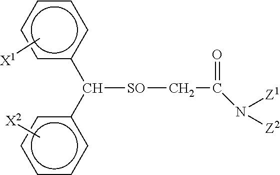
wherein
X¹ and X² may be the same or different, and are H, Cl, or F;
Z¹ and Z² are the same or different and are H, —CH₃, —CH(CH₃)₂, or —C(CH₃)₃, with the proviso that at least one Z¹ and Z² is H, or a pharmaceutically acceptable salt thereof.

13. The method of claim 8, wherein the benzhydrylsulfinylacetamide wakefulness promoting agent is benzhydrylsulfinylacetamide, or a pharmaceutically acceptable salt thereof.

14. The method of claim 14, wherein the benzhydrylsulfinylacetamide wakefulness promoting agent is present in an amount of 50-500 mg.

15. A method for alleviating side-effects associated with the administration of cognition effecting pharmaceuticals comprising:
administering a benzhydrylsulfinylacetamide wakefulness promoting agent and an amount of sodium chloride to a subject having a side-effect from a cognition effecting pharmaceutical;
wherein the amount of sodium chloride is effective to increase mean arterial blood pressure of the subject.

16. The method of claim 15, wherein the amount of sodium chloride is 0.25 grams or more.

17. The method of claim 15, wherein the amount of sodium chloride is effective to raise the mean arterial blood pressure of the subject by 10 mm Hg or less.

18. The method of claim 15, wherein the benzhydrylsulfinylacetamide wakefulness promoting agent has the formula 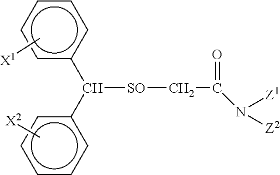
wherein
X¹ and X² may be the same or different, and are H, Cl, or F;
Z¹ and Z² are the same or different and are H, —CH₃, —CH(CH₃)₂, or —C(CH₃)₃, with the proviso that at least one Z¹ and Z² is or a pharmaceutically acceptable salt thereof.

19. The method of claim 15, wherein the benzhydrylsulfinylacetamide wakefulness promoting agent is benzhydrylsulfinylacetamide, or a pharmaceutically acceptable salt thereof.

20. The method of claim 15, wherein the benzhydrylsulfinylacetamide wakefulness promoting agent is present in an amount of 50-500 mg.
